(19) United States
(12) Patent Application Publication
(10) Pub. No.: US 2018/0296385 A1
(43) Pub. Date: Oct. 18, 2018

(54) System, Method, and Apparatus for Bowel Care

(71) Applicants: Elizabeth P. Ohler, Seffner, FL (US); Mark K. Ohler, Seffner, FL (US)

(72) Inventors: Elizabeth P. Ohler, Seffner, FL (US); Mark K. Ohler, Seffner, FL (US)

(21) Appl. No.: 16/010,551

(22) Filed: Jun. 18, 2018

Related U.S. Application Data
(63) Continuation-in-part of application No. 15/007,376, filed on Jan. 27, 2016, now Pat. No. 10,022,108.

(51) Int. Cl. A61F 5/451; A61F 5/443

(52) U.S. Cl. CPC A61F 5/451; A61F 5/4404; A61F 5/443

(57) ABSTRACT

An apparatus for human waste collection includes a containment component that includes an enclosed area that has a female receptacle for capturing of excrement. The containment component having a female receptacle interfaced to the enclosed area. A patient component is included for removably attaching to a user. The patient component has an opening for interfacing with an anus of the user and the opening is in fluid communication with a guide barrel. The guide barrel is configured to mate with the female receptacle of the containment component. The patient component has an adhesive material for removably adhering to the user at a location of the anus of the user. The containment component and the patient component removably join to form a single united structure

### CROSS-REFERENCE TO RELATED APPLICATION

This application is a continuation-in-part of U.S. patent application Ser. No. 15/007,376, filed Jan. 27, 2016, the disclosure of which is hereby incorporated by reference.

### FIELD

The present invention relates generally to fecal management devices, particularly to human waste collection devices and, more particularly, to disposable bags for use with such devices, and all being suitable for bedridden and incontinent patients.

### BACKGROUND

Bedridden and incontinent patients typically require continuous care to dispose of human waste products. Typically, bedridden patients may relieve themselves into a separate receptacle that is then replaced by a caretaker. As the patient has to adjust their position to use the receptacle, such a method of relief may be cause discomfort to the patient for physical reasons as well as for reasons of embarrassment.

Patients unable to move, as well as incontinent patients, are typically incapable of using a separate receptacle for relief. In such cases, human waste products are typically collected on an absorbent pad or similar device that may be fastened to the patient's bed or to the patient themselves. However, such devices are incapable of preventing human waste products from contacting the skin of the patient due to compression of the absorbent pad by the patient's weight. Consequently, the patient may develop pressure ulcers from continued contact with the waste products. Additionally, the necessity of having a caretaker change an unclean absorbent pad may cause further embarrassment for the patient.

Other alternatives for waste collection for bedridden and incontinent patient include waste collection receptacles that may be inserted into a cavity defined in a mattress while the patient is relieving themselves. However this alternative does not prevent the human waste products from contacting the patient's skin due to compression of the mattress and may be difficult to remove while the patient remains on the bed. A similar alternative involves adding a mattress overlay device to address the problem of mattress compression; however for reasons of patient comfort and other factors the device needs to be installed when the patient is ready to relieve themselves and removed immediately following relief, making such a device unsuitable for incontinent patients. Another alternative involves inflating a mattress and inserting a waste collection receptacle into a cavity defined in the mattress when the mattress is inflated; however, this device must similarly be inserted when the patient is ready for relief and removed immediately thereafter, thereby making continuous collection of human waste products impossible.

As such, it may be appreciated that there continues to be a need for a new and improved human waste collection device as set forth by the instant invention which addresses the problems of ease of use as well as effectiveness in construction in providing the ease to collect the human waste into a collection bag for bedridden and incontinent patients, and in this respect, the present invention substantially fulfills this need.

What is needed is a system that will collect human waste with minimal exposure of a user to such human waste.

### SUMMARY

In one embodiment, a human waste collection device is disclosed including a first component comprising an enclosed area that includes a female receptacle for capturing of excrement material and a second component for removably attaching to a user. The second component has an opening for interfacing with an anus of the user. The opening is in fluid communication with a guide barrel that is configured to mate with the female component of the first component. The second component has an adhesive material for removably adhering to the user at a location of the anus of the user. The first component and the second component removably join to each other form a single united structure. The guide barrel is of any shape that will accept and flow excrement from the user to the containment area.

In another embodiment, an apparatus for human waste collection is disclosed including a containment component that includes an enclosed area that has a female receptacle for capturing of excrement material. The containment component having a female receptacle interfaced to the enclosed area. A patient component is included for removably attaching to a user. The patient component has an opening for interfacing with an anus of the user and the opening is in fluid communication with a guide barrel. The guide barrel is configured to mate with the female receptacle of the containment component. The patient component has an adhesive material for removably adhering to the user at a location of the anus of the user. The containment component and the patient component removably join to form a single united structure.

In another embodiment, a method of collecting human waste is disclosed including affixing a patient component of a human waste collection device to a user, an opening of the patient component interfacing with an anus of the user. The method includes connecting a female receptacle of a containment component of the human waste collection device to a guide barrel of the patient component. The female receptacle of the patient component is in fluid communication with a guide barrel and the opening of the patient component mating with the guide barrel. The containment component has an enclosed area for collection of excrement. The enclosed area is interfaced to the female receptacle for capturing of the excrement.

In view of the foregoing disadvantages inherent in the known types of fecal management devices now present in the prior art, the present invention provides a human waste collection device wherein the same provides a bowel care collection bag to collect human waste for bedridden and incontinent patients and use on process of various components employed in the procedure. As such, the general purpose of the present invention, which will be described subsequently in greater detail, is to provide a new and improved fecal management device which has all the advantages of the prior art and none of the disadvantages.

Accordingly, it is an aspect of the present invention to provide a human waste collection device utilized for collecting feces from people unable to control their bowel movement, such as bedridden and incontinent patients. For the purpose, the bowel care collection bag is provided.

In another aspect, the present invention provides a feces containment device that adheres to the human skin while in use, providing a sanitized and hygienic environment.

Additionally, in some embodiments, the present invention includes any or all of a guide barrel, a viewing window, gas release holes and an occult blood testing strip.

Further aspects provide a hygienic, sealable, bio-degradable, portable, simple and convenient to use human waste collection device.

It is still another aspect of the present invention is to provide a new and improved human waste collection device which may be easily and efficiently manufactured and marketed.

It is a further aspect of the present invention is to provide a new and improved human waste collection device which is of a durable and reliable construction.

An even further aspect of the present invention is to provide a new and improved human waste collection device which is susceptible to a low cost of manufacture with regard to both materials and labor, and which accordingly is then susceptible of low prices of sale to the consuming public, thereby making such devices economically available to the buying public.

Other aspects of the present invention will become apparent from time to time throughout the specification as hereinafter related.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention can be best understood by those having ordinary skill in the art by reference to the following detailed description when considered in conjunction with the accompanying drawings in which:

FIG. 1 is a perspective view of the human waste collection device.

FIG. 2 is a graphical representation of the human waste collection device.

FIG. 3 is a perspective view of the human waste collection device.

FIG. 4 is a perspective view of the human waste collection device with a backflow check valve.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

In some embodiments, a human waste collection device utilized for collecting feces from people, such as bedridden and incontinent patients is disclosed. The device allows for continuous collection of human waste products while maintaining the bedridden and incontinent patient in a comfortable position.

In one embodiment, the present inventive human waste collection device helps to prevent the occurrence of irritation and infection resulting from the skin's frequent contact with fecal matters. In one embodiment, the continuous use of present inventive device eliminates the need of topical tropical ointments, cleansing solutions, disposable and washable bed pads, antibiotic creams or medicaments to eradicate the infection, skin protectants, disposable wipes and other related bowel care toiletries. In one embodiment, the present inventive device facilitates preventing human waste products from contacting the skin of the patient, thereby substantially reducing the occurrence of pressure ulcers. In one embodiment, the present device for collection of human waste products allow for an easy and efficient process of waste product disposal, thereby reducing the physical and emotional discomfort of the patient, and with careful containment of human waste into a well-designed bag, the contamination is diminished, and thus preventing the patient and caregiver from contracting the ailments.

With reference now to the drawings, a new and improved human waste collection device embodying the principles and concepts of the present invention are described herein.

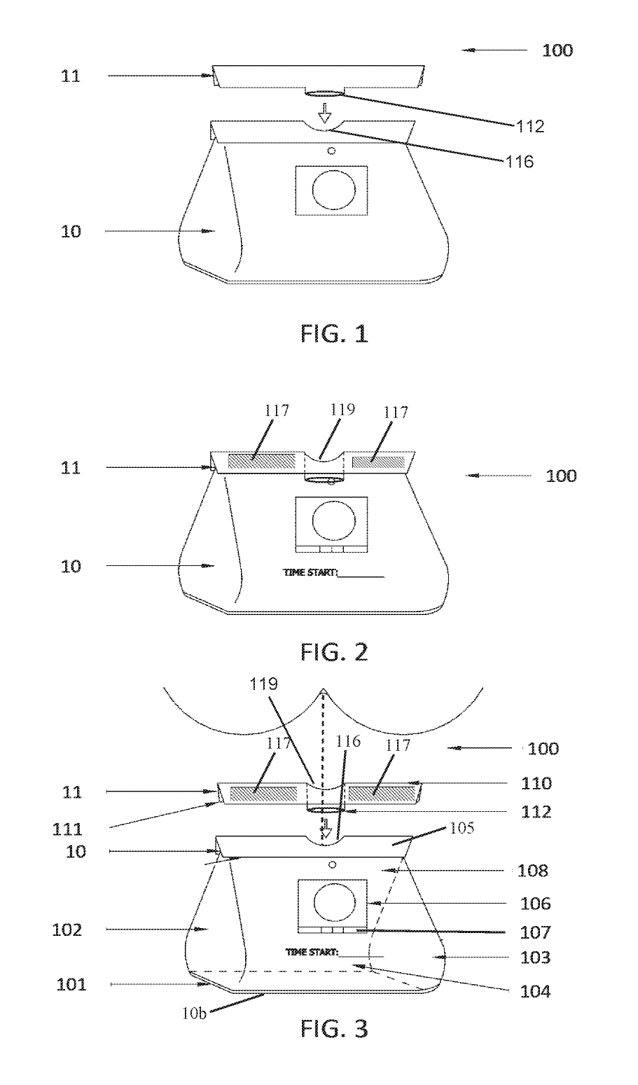

Referring to FIG. 1 and FIG. 2, the human waste collection device 100 is shown. The overall assembly of the waste collection device includes a first component 10 (containment component) for containing the fecal matter and a second component 11 (patient component), for removably affixing to the user (not shown). In general, the second component 11 attaches to the user by an adhesive 117, preferably an adhesive 117 that is strong enough to support both the first component 10 and the second component 11 during use, yet having a weak enough bond so as to be removable from the user without undue pain or tearing of the user's skin, for example an adhesive 117 such as is used for adhesive tape and bandages.

In FIG. 2, the second component 11 is shown with an opening 119 that interfaces with the anus of the user. The opening 119 is fluidly connected to a guide barrel 112. The guide barrel 112 interfaces with a female receptacle 116 when the first component 10 is connected to the second component 11, staying connected either through friction between the guide barrel 112 and the female receptacle 116 or through any other means including, but not limited to another adhesive, clips, hook and loop material, etc. It is preferred that the second component 11 include the guide barrel 112 and the first component 10 include the female receptacle 116 for proper flow of fluids (e.g. fecal matter), though it is equally anticipated that the second component 11 include the female receptacle 116 and the first component 10 include the guide barrel 112.

As shown in FIG. 3, the first component 10, includes a container portion (e.g. similar to a conventional “brown bag” like structure) comprising a base 101 (for example, shown generally rectangular), and sides 102 (shown generally triangular), a front side 103 and a rear side 104. The top 105 is shaped to mate with the bottom surface 111 of the second component 11. The top 105 of the first component 10 comprises the female receptacle 116 for receiving the guide barrel 112 of the second component 11. In some embodiments, the top 105 of the first component 10 comprises a second adherent material for bonding between the first component 10 and the second component 11.

In some embodiments, the first component 10 includes a viewing window 106 at the front side 103 that allows the caregiver a clear view of the amount of fluid (e.g. fecal matter) that has been deposited inside the bag.

In some embodiments, the first component 10 includes a built-in occult blood testing strip 107 that is fluidly interfaced to a location within the first component 10. The occult blood testing strip 107, after contact with fecal matter, provides for showing an indication of the presence of blood in the stool should that be the case.

In some embodiments, the first component 10 includes at least one gas release hole 108 which permits the first component 10 to be opened to permit gases accumulated therein to be expelled, or fecal matter to be removed while the bag remains on the user.

In some embodiments, the first component 10 includes a pocket 10b for containing a plastic bag (not shown). The plastic bag is easily pulled out from a pocket 10b and then is used to contain a first component 10 that is full to prevent splatters and leakage and to keep the waste products contained therein, prior to proper disposal.

The second component 11 includes the adhesive 117 on a top surface 110 (all or part of the top surface 110). The adhesive 117 removably adheres to the buttock area in the vicinity of the human anus, holding the second component 11 so that the opening 119 of the second component 11 is in fluid communication with the anus of the user for receiving excrement from the anus of the user. In some embodiments, a bottom surface 111 of the second component 11 also includes an adherent material for maintaining a connection between the first component 10 and the second component.

In use, a caregiver adheres the second component 11 to the user, aligning the opening 119 with the anus of the user. Once the second component 11 is installed on the user, the first component 10 is mated and affixed to the second component 11 (either by friction or by an adhesive layer). After the first component 10 fills with fecal matter, the first component 10 is dislodged from the second component 11 and disposed (e.g. utilizing the plastic bag previously discussed) and a new first component 10 is mated and affixed to the second component 11 (either by friction or by an adhesive layer), as needed.

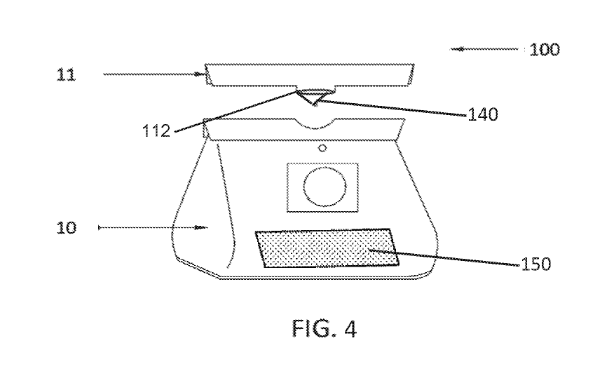

Referring now to FIG. 4, in some embodiments, the human waste collection device 100 is shown with a check valve 140 interfaced to or within the guide barrel 112. Although any check valve 140 is anticipated, a duck-billed type of check valve 140 is shown, though flapper valves or any one-flow-direction valve is anticipated.

Also, in some embodiments as shown in FIG. 4, the first component 10 includes an absorbent material 150 to absorb liquid excrement as fecal matter isn't always solid or semi-solid. Any absorbent material is anticipated, for example, a super-absorbent polymer as used in hygiene products and disposable diapers.

As may now be appreciated from the above description and reference to the accompanying drawings, the present invention provides a human waste collection device which may be utilized by caregivers to ease the collection of the human waste into a collection bag for any user.

It is to be understood that the above description is intended to be illustrative, and not restrictive. For example, the above-discussed embodiments may be used in combination with each other. Many other embodiments will be apparent to those of skill in the art upon reviewing the above description.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A human waste collection device, comprising:
a first component comprising an enclosed area that includes a female receptacle for passing an excrement into the enclosed area; and
a second component for removably attaching to a user, the second component comprising an opening for interfacing with an anus of the user, the opening in fluid communication with a guide barrel configured to mate with the female component of the first component, the second component having an adhesive material for removably adhering to the user at a location of the anus of the user;
wherein the first component and the second component removably join to form a single united structure.

2. The human waste collection device of claim 1, further comprising an adhesive between the first component and the second component, the adhesive providing holding force between the first component and the second component and for removal of the first component from the second component without need to remove the second component from the user.

3. The human waste collection device of claim 1, the first portion further comprising an occult for blood testing strip, the occult blood testing strip interfaced to an interior of the enclosed area for contact with the excrement that is held within the enclosed area, the occult blood testing strip visible from outside of the enclosed area for direct reading and determination of a presence of a blood in the excrement.

4. The human waste collection device of claim 1, the first portion further comprising at least one gas release hole interfaced to the enclosed area, the at least one gas release hole permitting the enclosed area to be opened to permit gases accumulated therein to be expelled or the excrement to be removed while the first component remains connected to the second component.

5. The human waste collection device of claim 1, the first portion further comprising another adhesive material between a bottom side of the second component and a top side of the first component.

6. The human waste collection device of claim 1, the first portion further comprising an absorbent material positioned within the enclosed area.

7. The human waste collection device of claim 1, the first portion further comprising a check valve interfaced to the guide barrel, the check valve directed so that the excrement can flow into the enclosed area, but the excrement already within the enclosed area cannot flow out of the enclosed area when the first portion is interfaced to the second portion.

8. An apparatus for human waste collection, the apparatus comprising:
a containment component comprising an enclosed area that includes female receptacle for capturing of an excrement, the containment component having a female receptacle; and
a patient component for removably attaching to a user, the patient component comprising an opening for interfacing with an anus of the user, the opening in fluid communication with a guide barrel, the guide barrel configured to mate with the female receptacle of the containment component, the patient component having an adhesive material for removably adhering to the user at a location of the anus of the user;
wherein the containment component and the patient component removably join to form a single united structure.

9. The apparatus for human waste collection of claim 8, further comprising an adhesive between the patient component and the containment component, the adhesive providing holding force between the patient component and the containment component and for removal of the patient component from the containment component without need to remove the patient component from the user.

10. The apparatus for human waste collection of claim 8, the containment component further comprising an occult for blood testing strip, the occult blood testing strip interfaced to an interior of the enclosed area for contact with the excrement that is held within the enclosed area, the occult blood testing strip visible from outside of the enclosed area for direct reading and determination of a presence of a blood in the excrement.

11. The apparatus for human waste collection of claim 8, the first portion further comprising at least one gas release hole interfaced to the enclosed area, the at least one gas release hole permitting the enclosed area to be opened to permit gases accumulated therein to be expelled or fecal matter to be removed while the containment component remains connected to the patient component.

12. The apparatus for human waste collection of claim 8, the containment portion further comprising another adhesive material between a bottom side of the patient component and a top side of the containment component.

13. The apparatus for human waste collection of claim 8, the containment portion further comprising an absorbent material positioned within the enclosed area.

14. The apparatus for human waste collection of claim 8, the patient portion further comprising a check valve interfaced to the guide barrel, the check valve directed so that the excrement can flow into the enclosed area, but the excrement already within the enclosed area cannot flow out of the enclosed area when the patient portion is interfaced to the containment portion.

15. A method of collecting human waste, the method comprising:
affixing a patient component of a human waste collection device to a user, an opening of the patient component interfacing with an anus of the user; and
connecting a female receptacle of a containment component of the human waste collection device to a guide barrel of the patient component, the female receptacle of the patient component being in fluid communication with the guide barrel and the female receptacle of the patient component mating with the guide barrel, the containment component having an enclosed area for collection of the excrement;
whereas the enclosed area is fluidly interfaced to the female receptacle for capturing of the excrement.

16. The method of collecting human waste of claim 15, the method further comprising:
while the patient component of the human waste collection device remains affixed to the user:
removing the female receptacle of the containment component of the human waste collection device from the guide barrel of the patient component; and
connecting a second female receptacle of a second containment component of a second human waste collection device to the guide barrel of the patient component.

17. The method of collecting human waste of claim 15, the method further comprising:
while the patient component of the human waste collection device remains affixed to the user:
opening a gas release hole, the gas release hole interfaced to the enclosed area of the containment portion; and
removing a portion of the excrement from the enclosed area of the containment component.
